(19) United States
(12) Patent Application Publication
(10) Pub. No.: US 2011/0171279 A1
(43) Pub. Date: Jul. 14, 2011

(54) POLYETHYLENIMINE BIOCIDES

(75) Inventors: Bret Ja Chisholm, West Fargo, ND (US); Shane J. Stafslien, West Fargo, ND (US); Alexander John Kugel, Saint Paul, MN (US)

(73) Assignee: NDSU RESEARCH FOUNDATION, Fargo, ND (US)

(21) Appl. No.: 12/917,618

(22) Filed: Nov. 2, 2010

Related U.S. Application Data
(60) Provisional application No. 61/257,264, filed on Nov. 2, 2009.

(51) Int. Cl. A01N 33/02; C07C 213/00; A01N 25/00; A01N 25/34; A01P 1/00

(52) U.S. Cl. 424/411; 564/347; 514/651; 424/405

(57) ABSTRACT

A family of polyethylenimine biocides of formula (I):

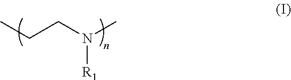

wherein R₁ is hydrogen, a branched ethylenimine, a tethered antimicrobial moiety, or a branched ethylenimine having a tethered antimicrobial moiety is described herein. The polyethylenimine biocides may be used in a variety of consumer products, coatings, coating compositions, and medical devices to prevent growth of microorganisms.

### CROSS-REFERENCE TO RELATED APPLICATIONS

This application claims priority under 35 U.S.C. §119(e) to U.S. Provisional Patent Application No. 61/257,264 filed on Nov. 2, 2009. The contents of this application are hereby incorporated by reference in their entirety.

### STATEMENT REGARDING FEDERALLY SPONSORED RESEARCH AND DEVELOPMENT

This invention was made with government support under grant N00014-07-1-1099 awarded by The Office of Naval Research (ONR). The Government has certain rights in the invention.

### FIELD OF THE INVENTION

The invention relates to antimicrobial or biocide compositions and coatings. It is desired to eliminate or prevent the growth of unwanted organisms, for example, to combat the spread of infectious disease in hospitals, mold and mildew on architectural surfaces, biofouling on marine vessels, pathogenic microorganisms in the home, and pathogenic microorganisms in food and consumer products. Due to the significance of the microorganism problem, new antimicrobial materials are needed.

### SUMMARY

In one embodiment, the invention provides, among other things, a polymeric biocide of formula (I):

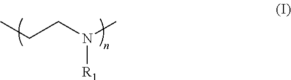

wherein each R₁ is independently H; formula (II):

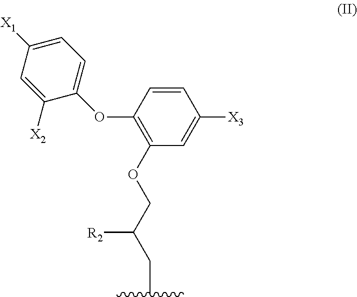

wherein X₁, X₂, X₃, are independently H or halogen and R₂ is OH, NHOH, NH₂, or C₁-C₄ alkyl alcohol; formula (III):

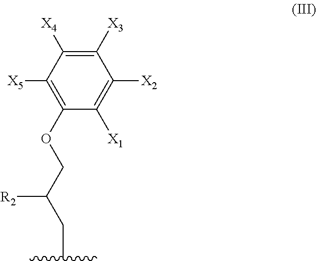

wherein X₁-X₅ are independently H or halogen and R₂ is OH, NHOH, NH₂, or C₁-C₄ alkyl alcohol; or (CH₂)₂NR₃R₄ wherein R₃ and R₄ are independently H, (CH₂)₂NH₂, formula (II) or formula (III); and n=5-50,000, wherein at least one R₁ or R₃ or R₄ is either formula (II) or formula (III). In one embodiment X₁, X₂, X₃, X₄, or X₅, is chlorine. The polymeric biocides may be incorporated into any number of products including cosmetics, lotions, creams, etc. The polymeric biocides may also be incorporated into coatings such as paints, especially marine paints. Optionally, the coatings may have crosslinkers. The polymeric biocides may be effective against a number of microorganisms, including, but not limited to, Staphylococcus epidermidis, Escherichia coli, Navicula incerta, Cellulophaga lytica, Halomonas pacifica, Pseudoalteromonas atlantica, Cobetia marina, Candida albicans, Clostridium difficile, and Listeria monocytogenes.

In another embodiment, the invention provides, among other things, a polymeric biocide formed by reacting an ethylenimine polymer with 2-((5-chloro-2-(2,4-dichlorophenoxy)phenoxy)methyl)oxirane.

In another embodiment, the invention provides, among other things, a polymeric biocide formed by reacting an ethylenimine polymer with 2-((2,4,6-trichlorophenoxy)methyl)oxirane.

Other aspects of the invention will become apparent by consideration of the detailed description and accompanying drawings.

### DETAILED DESCRIPTION

The invention provides a family of polyethylenimine biocides of formula (I):

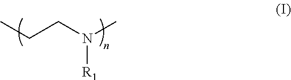

wherein R₁ is hydrogen, a branched ethylenimine, a tethered antimicrobial moiety, or a branched ethylenimine having a tethered antimicrobial moiety. The polyethylenimine biocides must have at least one tethered antimicrobial moiety to be effective as biocides. The antimicrobial moieties of the invention include formula (II):

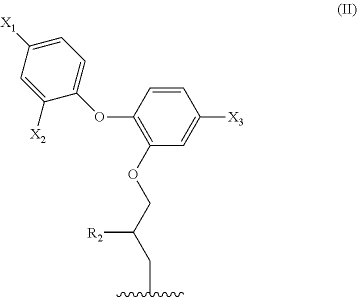

wherein X₁, X₂, X₃, are independently H or halogen and R₂ is OH, NHOH, NH₂, or C₁-C₄ alkyl alcohol and formula (III):

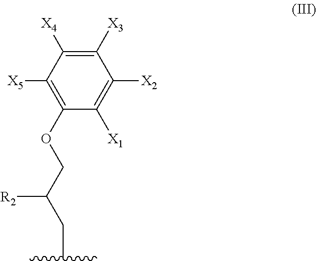

wherein X₁-X₅ are independently H or halogen and R₂ is OH, NHOH, NH₂, or C₁-C₄ alkyl alcohol. In some embodiments, the tethered antimicrobial moiety is

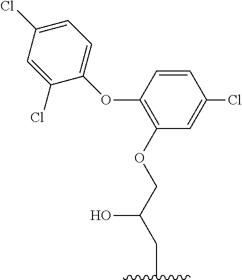

in other embodiments, the tethered antimicrobial moiety is

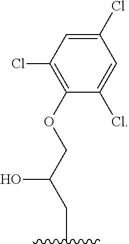

Polyethylenimine biocides of the invention are effective in inhibiting the growth of many microorganisms. As used herein, microorganisms includes single-cell and multi-cell bacteria, fungi, parasites, protozoans, archaea, protests, amoeba, viruses, diatoms, and algae. Microorganisms whose growth may be inhibited by polyethylenimine biocides of the invention include, but are not limited to, Staphylococcus aureus, Staphylococcus epidermidis, Streptococcus faecalis, Bacillus subtilis, Salmonella chloraesius, Salmonella typhosa, Escherichia coli, Mycobacterium tuberculosis, Pseudomonas aeruginosa, Aerobacter aerogenes Saccharomyces cerevisiae, Candida albicans, Aspergillus niger, Aspergillus flares, Aspergillus terreus, Aspergillus verrucaria, Aureobasidium pullulans, Chaetomium globosum, Penicillum funiculosum, Trichophyton interdigital, Pullularia pullulans, Trichoderm sp. madison P-42, and Cephaldascus fragans; Chrysophyta, Oscillatoria bometi, Anabaena cylindrical, Selenastrum gracile, Pleurococcus sp., Gonium sp., Volvox sp., Klebsiella pneumoniae, Pseudomonas fluorescens, Proteus mirabilis, Enterobacteriaceae, Acinetobacter spp., Pseudomonas spp., Candida spp., Candida tropicalis, Streptococcus salivarius, Rothia dentocariosa, Micrococcus luteus, Sarcina lutea, Salmonella typhimurium, Serratia marcescens, Candida utilis, Hansenula anomala, Kluyveromyces marxianus, Listeria monocytogenes, Serratia liquefasciens, Micrococcus lysodeikticus, Alicyclobacillus acidoterrestris, MRSA, Bacillus megaterium, Desulfovibrio sulfuricans, Streptococcus mutans, Cobetia marina, Enterobacter aerogenes, Enterobacter cloacae, Proteus vulgaris, Proteus mirabilis, Lactobacillus plantarum, Halomonas pacifica, Ulva linza, and Clostridium difficile. Polyethylenimine biocides of the invention may inhibit the growth of small colonies of microorganisms, as well as biofilms.

The polyethylenimine biocides of the invention are typically formed by reacting a polyethylenimine composition with an epoxy triclosan or an epoxy trichlorophenol in the presence of heat. A suitable epoxy triclosan may be 2-((5-chloro-2-(2,4-dichlorophenoxy)phenoxy)methyl)oxirane

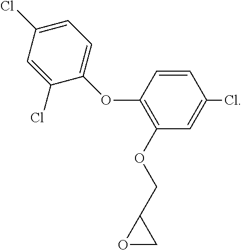

A suitable epoxy trichlorophenol may be 2-((2,4,6-trichlorophenoxy)methyl)oxirane

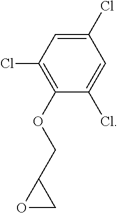

Exemplary synthetic methods are shown in EXAMPLES 1, 2, and 9 below. Polyethylenimine compositions suitable for incorporation with the invention range from ethylenimine oligomers, with only about 10 repeat units, to large polymers having 50,000 or more repeat units. The polyethylenimines may be linear, branched, or dendritic. Suitable polyethylenimines are available from a number of suppliers, including, but not limited to, Sigma-Aldrich (St. Louis, Mo.). Depending upon the ratio of polyethylenimine to tethered antimicrobial moiety, and the nature of the polyethylenimine, the resultant polyethylenimine biocide may have a glass transition temperature (T_{g}) between about −100° C. and 50° C., typically between about −80° C. and 20° C., more typically between about −60° C. and 0° C. Polyethylenimine biocides of the invention may have a 1:1 mole ratio of ethylenimine monomers to antimicrobial moieties, typically greater than about a 5:1 mole ratio of ethylenimine monomers to antimicrobial moieties, more typically greater than about a 10:1 mole ratio of ethylenimine monomers to antimicrobial moieties. The ability to manipulate the glass transition temperature of the polyethylenimine biocides allows the polyethylenimine biocides to be incorporated into many different products. Additionally, because some of the polyethylenimine biocides are water soluble, they can be incorporated into aqueous systems. Other polyethylenimine biocides are more hydrophobic, and can be incorporated into lipid systems, e.g., creams.

Polyethylenimine biocides of the invention which remain liquid at room temperature are suitable for incorporation into a variety of consumer products, including cosmetics, creams, lotions, toothpastes, shampoos, anti-perspirants, etc. In particular, polyethylenimine biocides are suitable for incorporation into cosmetics including, but not limited to, mascara, foundation, blush, lipstick, eye shadow, eyeliner, concealer, wrinkle cream, and moisturizers. As necessary, any number of additional components may be added to polyethylenimine biocide compositions of the invention to achieve the desired smell, texture, color, or fragrance. For example, compositions of the invention may additionally comprise emulsifiers, stabilizers, thickeners, humectants, or plasticizers. Compositions of the invention may also comprise fragrances, pigments, and dyes.

Biocidel polymers of the invention may be incorporated into compositions comprising additional biocides, including, but not limited to, 2-methylthio-4-butylamino-6-cyclopropylamine-s-triazine (Irgarol 1051), 2,3,5,6-tetrachloro-4-(methylsulfonyl)pyridine (TCMSpyridine), (2-thiocyanomethylthio)benzothiazole (TCMTB), (4,5-dichloro-2-n-octyl-4-isothazolin-3-one) (SEA-NINE™ 211), (2,4,5,6-tetrachloroisophthalonitrile) (chlorothalonil), 3-(3,4-dichlorophenyl)1,1-dimethylurea (diuron), 2,4,6-trichlorophenylmaleimide, bis(dimethylthiocarbamoyl)disulfide (Thiram), 3-iodo-2-propynyl butylcarbamate, N,N-dimethyl-N′-phenyl(N′-fluorodichloromethylthiosulfamide (Dichlorofluanid), N-(fluorodichloromethylthio)phthalimide, diiodomethyl-p-tolysulfone, 5,6-dihydroxy-3-(2-thienyl)-1,4,2-oxathiazine, 4-oxide, 5,7-dichloro-8-hydroxy-2-methylquinoline, 2,5,6-tribromo-1-methylgramine, (3-dimethylaminomethyl-2,5,6-tribromo-1-methylindole)2,3-dibromo-N-(6-chloro-3-pyridyl)succinimide, thiazoleureas, 3-(3,4-dichlorophenyl)-5,6-dihydroxy-1,4,2-oxathiozine oxide, 2-trifluoromethyl-3-bromo-4-cyano-5-parachlorophenyl pyrrole, 2-bromo-4′-chloroacetanilide, 2,6-bis(2′,4′-dihydroxybenzyl)-4-methylphenyl, 2,2-bis(3,5-dimethoxy-4-hydroxyphenyl)propane; acylphloroglucinols, such as 2,6-diacyl-1,3,5-trihydroxybenzene; guanidines, such as 1,3-dicyclohexyl-2-(3-chlorophenyl)guanidine; alkylamines, such as auryldimethylamine; dialkylphosphonates, such as phosphoric acid di(2-ethylhexylester); alkyl haloalkyl disulfides, such as n-octylchloromethyl disulfide and 4,5-dicyano-1,3-dithiole-2-thione; enzymes, such as endopeptidases, glucose oxidases, and lysozymes; antimicrobial peptides, such as Polymyxin B, EM49 and bacitracin; and natural products, such as vancomycin and chitosan. Metal biocides, metal salt biocides, and metal oxide biocides may also be incorporated into compositions of the invention. Metals suitable for use include, but need not be limited to, silver, copper, zinc, titanium, and tin.

It is additionally possible to prepare coatings and coating compositions comprising polyethylenimine biocides of the invention. Such coatings are beneficial for protecting surfaces from attachment of microorganisms, or for reducing the growth of microorganisms on these surfaces, or for preventing the spread of microorganisms between people who contact those surfaces. Surfaces suitable for coating with polyethylenimine biocides include medical surfaces, marine surfaces, and household surfaces. Marine surfaces include, but are not limited to, boat or ship hulls, anchors, docks, jetties, sewage pipes and drains, fountains, water-holding containers or tanks, and any other surface in contact with a freshwater or saltwater environment. The surface may be a medical surface. Medical surfaces include, but are not limited to, implants, medical devices, examination tables, and instrument surfaces. Implants and medical devices may include, but are not limited to, prosthetic heart valves, urinary catheters, stents, and orthopedic implants. The surface may also be a household surface. Household surfaces include, but are not limited to, countertops, sink surfaces, cupboard surfaces, and shelf surfaces.

Coatings according to the invention may comprise additional components to achieve desired properties, including abrasion-resistance improvers, adhesion promoters, anti-blocking agents, anti-cratering agents, anti-crawling agents, anti-float agents, anti-flooding agents, anti-foaming agent, anti-livering agent, anti-marring agent, antioxidants, block resistant additive, brighteners, burnish-resistant additives, catalysts, corrosion-inhibitors, craze-resistance additive, deaerators, defoamers, dispersing agent, matting agents, flocculants, flow and leveling agents, gloss improvers, hammer-finish additives, hindered amine light stabilizers, intumescent additives, luminescent additives, mar-resistance additives, masking agents, rheology modifiers, slip-aids, spreading agents, static preventative, surface modifiers, tackifiers, texturizing agents, thixotropes, tribo-charging additive, UV absorbers, waxes, wet edge extenders, or wetting agents. Most additives are usually less than 5% by weight or less of the final coating formulation. Pigments and fillers may be higher-possibly 5%-50% by weight.

In one embodiment, polyethylenimine biocides may be incorporated into coatings for knobs, handles, rails, poles, countertops, sinks, and faucets. In some embodiments the polyethylenimine biocides may be incorporated into paints, such as marine paints to inhibit biofouling of surfaces. In other embodiments, the polyethylenimine biocides may be incorporated into a polymer coating or resin which has inherent antimicrobial properties. In some embodiments, the polyethylenimine biocides may be incorporated into a copolymer to achieve desired coating properties. Copolymers may include block copolymers, including diblock, triblock, etc. In some embodiments, the polyethylenimine biocides may be crosslinked to form stronger and harder coatings. Crosslinking the biocide may enable longer lasting antimicrobial compositions, materials, coatings, etc.

In some embodiments, the polyethylenimine biocides may be crosslinked with other polymers, such as epoxy-functional polyethylene glycol (PEG), to produce hydrogels. In general, any hydrophilic compound with at least two functional groups capable of reacting with amines (e.g., epoxides and isocyanates) may be used as a crosslinker. Such hydrogels may be useful for incorporation into medical devices where long-lasting antimicrobial properties are beneficial. For example, the hydrogels may be incorporated into wound dressings. The antimicrobial hydrogels would be non-irritating to the wound, would absorb wound exudate, and, due to the inherently antimicrobial properties, enhance the sterile environment around the wound.

It is to be understood that the invention is not limited in its application to the details of construction and the arrangement of components set forth in the following description. The invention is capable of other embodiments and of being practiced or of being carried out in various ways. Also, it is to be understood that the phraseology and terminology used herein is for the purpose of description and should not be regarded as limiting.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the invention.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Further, no admission is made that any reference, including any patent or patent document, cited in this specification constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinency of any of the documents cited herein.

### EXAMPLES

### Example 1

### Epoxidized Triclosan (ETCS) Synthesis

80 g of isopropanol (Sigma-Aldrich, St. Louis, Mo.) and 19.4 g (0.346 mol) potassium hydroxide (Sigma-Aldrich) were charged to a one liter, three-neck, round-bottom flask equipped with a magnetic stir bar, and the mixture stirred at room temperature until the potassium hydroxide dissolved. In an 800 mL glass beaker, 100 g (0.346 mol) of triclosan (TCS) (Alfa Aesar, Ward Hill, Mass.) was dissolved at room temperature in 250 g of isopropanol using magnetic stirring. The TCS solution was subsequently added to the round-bottom flask containing the isopropanol/potassium hydroxide solution. The flask was then placed in a temperature-controlled silicone oil bath and equipped with a condenser and a 250 mL addition funnel. A thermocouple was placed into the reaction flask and the temperature controller set at 60° C.

Once the temperature had equilibrated, 95.9 g (1.036 mol) of epichlorohydrin (Sigma-Aldrich) was added dropwise to the solution over the course of 5 minutes using the addition funnel. During the course of the reaction, a precipitate (potassium chloride) was formed. The reaction was allowed to run for 16 hours. Upon completion of the reaction, the reaction mixture was transferred to a one-liter, single-neck, round-bottom flask and then placed in a rotary evaporator at reduced pressure for two hours to remove unreacted epichlorohydrin. Further purification was done using solvent extraction with water and a 1:1 mixture of hexanes (Sigma-Aldrich) and toluene (Sigma-Aldrich). The organic phase was washed four times with water and dried over magnesium sulfate (Sigma-Aldrich). Remaining solvent was removed at reduced pressure on a rotary evaporator and the clear viscous liquid product was collected (yield: 88%). The purified ETCS was characterized using proton nuclear magnetic resonance spectroscopy (¹H NMR) and high performance liquid chromatography (HPLC).

### Example 2

### Triclosan Polyethylenimine Formulations

Ten grams of an oligomer mixture of ethylenimine having an average Mₙ=423 (PEI423) (Sigma-Aldrich) and 5.1 g of ETCS prepared according to EXAMPLE 1 were dissolved at room temperature in 80 g of chloroform (VWR Scientific, West Chester, Pa.) using a 250 mL single-neck, round-bottom flask designed for use with a Radley Six-Place Carousel Reactor System (Radleys Discovery Technologies, Essex, U.K.) The flask was placed in the Radley Six-Place Carousel Reactor System under a nitrogen blanket and condenser with temperature controller set at 50° C. The reaction was allowed to run for 64 hours and reaction progress was monitored using ¹H NMR. Upon completion of the reaction, solvent was then removed at reduced pressure using a rotary evaporator. The PEI1423/ETCS polymer, a yellow viscous liquid, was collected and characterized using ¹H NMR and differential scanning calorimetry (DSC). The PEI423/ETCS polymer is soluble in water at 5 wt %.

Formulations 2-9 were synthesized using the same synthetic procedure as above, with the exception that PEI composition (i.e. molecular weight) and ETCS concentration were varied. That is, Formulations 1, 4, and 7 used PEI423 (above), Formulations 2, 5, and 8 used an ethylenediamine endcapped polyethylenimine having an average Mₙ≈600 (PEI600) (Sigma-Aldrich), and Formulations 3, 6, and 9 used a branched polyethylenimine having an average Mₙ≈10000 (PEI10000) (Sigma-Aldrich). For Formulation 10, 3.59 g of PEI423 and 1.45 g of epoxy triclosan were reacted in the absence of solvent. The mixture was heated at 65° C. for 72 hours, and reaction completion was confirmed by ¹H NMR. For Formulation 11, 2.82 g of PEI423 and 2.25 g of epoxy triclosan were reacted in the absence of solvent. The mixture was heated at 65° C. for 72 hours, and reaction completion was confirmed by ¹H NMR. For Formulation 12, 1.94 g of PEI423 and 3.06 g of epoxy triclosan were reacted in the absence of solvent. The mixture was heated at 65° C. for 72 hours, and reaction completion was confirmed by ¹H NMR.

The glass transition temperature (T_{g}) of the formulations produced by the reaction of PEI with ETCS were measured using differential scanning calorimetry. The T_{g}s obtained are shown in Table 1. The increase in polymer T_{g} with increasing modification of PEI with ETCS provides evidence of successful grafting of TCS moieties to the PEI polymer backbone.

TABLE 1
PEI423, PEI600, PEI10000, and Formulation 1-12 glass transition
temperatures (T_{g}), and compositions of Formulations 1-12.

| Compound | PEI component | Wt PEI/g | Wt ETCS/g | Tg/° C. |
|---|---|---|---|---|
| PEI423 | PEI423 | 100% | none | −60 |
| PEI600 | PEI600 | 100% | none | −52 |
| PEI10000 | PEI10000 | 100% | none | −48 |
| Formulation 1 | PEI423 | 10 | 5.1 | −53 |
| Formulation 2 | PEI600 | 10 | 5.1 | −37 |
| Formulation 3 | PEI10000 | 10 | 5.1 | −17 |
| Formulation 4 | PEI423 | 7.5 | 7.6 | −47 |
| Formulation 5 | PEI600 | 7.5 | 7.6 | −33 |
| Formulation 6 | PEI10000 | 7.5 | 7.6 | −1 |
| Formulation 7 | PEI423 | 5 | 10.2 | −38 |
| Formulation 8 | PEI600 | 5 | 10.2 | −27 |
| Formulation 9 | PEI10000 | 5 | 10.2 | 8 |
| Formulation 10 | PEI423 | 3.59 | 1.45 | −58 |
| Formulation 11 | PEI423 | 2.82 | 2.25 | −39 |
| Formulation 12 | PEI423 | 1.94 | 3.06 | −15 |

### Example 3

### Biocidal Activity of Formulations 1-9 Against Staphylococcus epidermidis

The antimicrobial properties of the Formulations were determined by measuring the minimum inhibitory concentration (MIC) or by using an adaptation of the MIC test. MIC is typically the lowest concentration of antimicrobial that completely inhibits growth over a set incubation period relative to a control containing no antimicrobial. Additional details of the MIC method can be found at Andrews, J. M., Journal of Antimicrobial Chemotherapy (2001) 48, 5-16, incorporated herein by reference in its entirety.

In accordance with the MIC test, working solutions for each Formulation were prepared by dissolving 100 mg of each Formulation in 10 mL of methanol (Sigma-Aldrich) to generate a 10 mg/mL solution. Next, 10 ml of tryptic soy broth was spiked with 200 μL of the mg/mL Formulation solution to achieve a final concentration of 0.2 mg/mL.

A series of dilutions of each Formulation was prepared by diluting the 0.2 mg/mL suspension of each Formulation in tryptic soy broth to generate concentrations of 100 μg/mL, 50 μg/mL, 25 μg/mL, 12.5 μg/mL 6.25 μg/mL, 3.13 μg/mL, 1.56 μg/mL and 0.78 μg/mL. 0.2 mL of each concentration of each Formulation (spiked with S. epidermidis) was added in triplicate to a 96-well plate. Additionally, 0.2 mL of tryptic soy broth without any S. epidermidis or Formulation and 0.2 mL of tryptic soy broth with S. epidermidis, but no Formulations, served as negative and positive growth controls, respectively. Plates were then placed in an incubator, at 37° C. for 24 hrs (with shaking), and then measured for growth by taking absorbance measurements at 600 nm with a multi-well plate spectrophotometer (Synergy HT microplate reader, BioTek Instruments, Winooski, Vt.). The efficacy of each Formulation was measured by determining the percent reduction in bacterial growth as a function of Formulation concentration. The results are shown in Table 2.

TABLE 2
Percent reduction in growth of Staphylococcus epidermidis
from serial dilutions of Formulations 1-9.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 0.00% | 12.50% | 14.55% | 24.32% | 24.20% | 18.52% | 24.77% | 8.98% |
| PEI600 | 19.65% | 23.52% | 25.23% | 26.44% | 28.25% | 26.63% | 20.33% | 53.42% |
| PEI10,000 | 13.11% | 19.65% | 23.44% | 19.07% | 20.92% | 17.99% | 16.02% | 63.11% |
| ETCS | 0.00% | 9.43% | 8.30% | 16.36% | 18.64% | 17.39% | 22.16% | 9.55% |
| TCS | 81.36% | 86.70% | 92.70% | 92.96% | 93.09% | 92.83% | 93.09% | 93.35% |
| Formulation 1 | 0.00% | 16.93% | 17.51% | 87.41% | 93.36% | 94.16% | 93.94% | 93.71% |
| Formulation 2 | 0.00% | 24.37% | 43.02% | 92.91% | 94.16% | 94.05% | 93.36% | 91.08% |
| Formulation 3 | 0.00% | 18.99% | 26.89% | 87.76% | 93.94% | 93.14% | 90.96% | 87.41% |
| Formulation 4 | 1.45% | 12.92% | 12.94% | 12.95% | 15.79% | 3.28% | 19.85% | 94.10% |
| Formulation 5 | 8.81% | 22.43% | 23.11% | 21.98% | 20.32% | 88.97% | 94.38% | 93.57% |
| Formulation 6 | 0.00% | 11.14% | 17.82% | 91.65% | 93.32% | 92.20% | 89.09% | 80.29% |
| Formulation 7 | 7.46% | 15.99% | 18.50% | 16.15% | 16.78% | 22.45% | 72.32% | 93.47% |
| Formulation 8 | 6.25% | 12.92% | 15.82% | 15.23% | 21.78% | 18.14% | 60.72% | 92.59% |
| Formulation 9 | 0.00% | 8.18% | 10.57% | 18.52% | 23.30% | 23.86% | 82.84% | 66.48% |

The results displayed in Table 2 show that the Formulations were biocidal toward S. epidermidis. Biocidel activity was dependent on polymer composition.

### Example 4

### Biocidal Activity of Formulations 1-9 against Escherichia coli

In accordance with the MIC test, working solutions for each Formulation were prepared by dissolving 100 mg of each Formulation in 10 mL of methanol (Sigma-Aldrich) to generate a 10 mg/mL solution. Next, 10 ml of Luria-Bertani broth was spiked with 200 μL of the mg/mL Formulation solution to achieve a final concentration of 0.2 mg/mL.

A series of dilutions of each Formulation were prepared by diluting the 0.2 mg/mL suspension of each Formulation in Luria-Bertani broth to generate concentrations of 100 μg/mL, 50 μg/mL, 25 μg/mL, 12.5 μg/mL 6.25 μg/mL, 3.13 μg/mL, 1.56 μg/mL and 0.78 μg/mL. 0.2 mL of each concentration of each Formulation (spiked with E. coli) was added in triplicate to a 96-well plate. Additionally, 0.2 mL of Luria-Bertani broth without any E. coli or Formulation and 0.2 mL of Luria-Bertani broth with E. coli, but no Formulations, served as negative and positive growth controls, respectively. Plates were then placed in an incubator, at 37° C. for 24 hrs (with shaking), and then measured for growth by taking absorbance measurements at 600 nm with a multi-well plate spectrophotometer (Synergy HT). The efficacy of each Formulation was measured by determining the percent reduction in bacterial growth as a function of Formulation concentration. The results are shown in Table 3.

TABLE 3
Percent reduction in growth of Escherichia coli
from serial dilutions of Formulations 1-9.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 0.00% | 0.00% | 0.00% | 0.00% | 0.00% | 2.85% | 6.49% | 0.00% |
| PEI600 | 0.00% | 4.57% | 0.00% | 2.47% | 6.27% | 11.12% | 20.23% | 22.12% |
| PEI10,000 | 0.00% | 7.38% | 7.25% | 6.90% | 26.70% | 29.81% | 57.07% | 59.61% |
| ETCS | 0.00% | 0.00% | 0.00% | 0.00% | 0.00% | 0.00% | 0.00% | 0.26% |
| TCS | 14.66% | 52.34% | 93.43% | 93.93% | 93.43% | 93.93% | 93.93% | 93.93% |
| Formulation 1 | 0.00% | 8.17% | 52.08% | 93.49% | 93.63% | 93.63% | 93.35% | 92.52% |
| Formulation 2 | 12.19% | 16.20% | 93.49% | 93.63% | 93.63% | 93.35% | 92.11% | 90.30% |
| Formulation 3 | 9.14% | 34.90% | 92.94% | 93.35% | 93.35% | 92.66% | 91.55% | 86.01% |
| Formulation 4 | 0.00% | 3.77% | 0.00% | 0.37% | 5.40% | 2.89% | 13.49% | 93.61% |
| Formulation 5 | 0.00% | 3.12% | 3.28% | 2.59% | 9.99% | 60.93% | 94.00% | 93.07% |
| Formulation 6 | 0.00% | 0.00% | 5.49% | 43.52% | 92.02% | 87.16% | 76.06% | 60.35% |
| Formulation 7 | 0.00% | 0.00% | 0.00% | 1.42% | 2.35% | 2.38% | 3.77% | 59.32% |
| Formulation 8 | 0.00% | 1.69% | 1.24% | 0.44% | 7.00% | 6.59% | 11.34% | 92.48% |
| Formulation 9 | 0.00% | 0.00% | 0.00% | 0.00% | 0.00% | 5.32% | 35.67% | 63.94% |

The results displayed in Table 3 show that the Formulations were biocidal toward E. coli. Biocidel activity was dependent on polymer composition.

### Example 5

### Biocidal Activity of Formulations 1-9 Against Navicula Incerta

In accordance with the MIC test, working solutions for each Formulation were prepared by dissolving 100 mg of each Formulation in 10 mL of methanol (Sigma-Aldrich) to generate a 10 mg/mL solution. Next, 10 ml of Guillard's F/2 medium was spiked with 200 μL of the 10 mg/mL Formulation solution to achieve a final concentration of 0.2 mg/mL.

A series of dilutions of each Formulation were prepared by diluting the 0.2 mg/mL suspension of each Formulation in Guillard's F/2 medium to generate concentrations of 100 μg/mL, 50 μg/mL, 25 μg/mL, 12.5 μg/mL 6.25 μg/mL, 3.13 μg/mL, 1.56 μg/mL and 0.78 μg/mL. 0.2 mL of each concentration of each Formulation (spiked with N. incerta) was added in triplicate to a 96-well plate. Additionally, 0.2 mL of Guillard's F/2 medium without any N. incerta or Formulation and 0.2 mL of Guillard's F/2 medium with N. incerta, but no Formulations, served as negative and positive growth controls, respectively. The 96-well plates were placed in an illuminated growth cabinet with a 16:8 light:dark cycle (photon flux density 33 μmol m⁻² s⁻¹) for 48 hrs at 18° C. and measured for chlorophyll fluorescence using a multi-well plate spectrophotometer (excitation: 360 nm; emission: 670 nm). The efficacy of each Formulation was measured by determining the percent reduction in diatom growth as a function of Formulation concentration. The results are shown in Table 4.

TABLE 4
Percent reduction in growth of Navicula incerta
from serial dilutions of Formulations 1-9.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 0.00% | 0.00% | 0.00% | 0.00% | 5.08% | 26.00% | 37.88% | 48.21% |
| PEI600 | 0.00% | 11.04% | 10.16% | 13.65% | 28.19% | 40.38% | 49.74% | 59.42% |
| PEI10,000 | 0.00% | 47.96% | 89.14% | 97.22% | 98.66% | 98.77% | 98.87% | 98.86% |
| ETCS | 0.00% | 0.00% | 0.00% | 0.00% | 34.14% | 89.92% | 91.40% | 93.37% |
| TCS | 14.26% | 22.00% | 84.51% | 91.29% | 94.57% | 93.55% | 94.21% | 94.95% |
| Formulation 1 | 0.00% | 11.30% | 26.52% | 70.54% | 86.88% | 93.01% | 94.92% | 95.59% |
| Formulation 2 | 0.00% | 0.10% | 3.63% | 24.23% | 70.97% | 90.42% | 94.28% | 95.29% |
| Formulation 3 | 0.04% | 11.07% | 24.24% | 55.84% | 92.23% | 94.54% | 95.02% | 95.43% |
| Formulation 4 | 0.00% | 0.00% | 0.00% | 0.00% | 15.12% | 28.81% | 74.91% | 98.69% |
| Formulation 5 | 0.00% | 0.00% | 0.00% | 4.17% | 17.99% | 38.98% | 77.03% | 98.69% |
| Formulation 6 | 0.00% | 0.00% | 40.15% | 75.12% | 90.95% | 94.09% | 94.72% | 94.96% |
| Formulation 7 | 0.00% | 0.00% | 0.00% | 0.00% | 4.70% | 26.28% | 50.03% | 98.16% |
| Formulation 8 | 0.08% | 13.83% | 6.71% | 5.11% | 4.72% | 12.43% | 82.20% | 98.45% |
| Formulation 9 | 0.00% | 0.00% | 0.00% | 0.00% | 0.00% | 10.03% | 41.55% | 65.15% |

The results displayed in Table 4 show that the Formulations were biocidal toward N. incerta. Biocidel activity was dependent on polymer composition.

### Example 6

### Biocidal Activity of Formulations 1-9 Against Cellulophaga lytica

In accordance with the MIC test, working solutions for each Formulation were prepared by dissolving 100 mg of each Formulation in 10 mL of methanol (Sigma-Aldrich) to generate a 10 mg/mL solution. Next, 10 ml of Guillard's F/2 medium was spiked with 200 μL of the 10 mg/mL Formulation solution to achieve a final concentration of 0.2 mg/mL.

A series of dilutions of each Formulation were prepared by diluting the 0.2 mg/mL suspension of each Formulation in Guillard's F/2 medium to generate concentrations of 100 μg/mL, 50 μg/mL, 25 μg/mL, 12.5 μg/mL 6.25 μg/mL, 3.13 μg/mL, 1.56 μg/mL and 0.78 μg/mL. 0.2 mL of each concentration of each Formulation (spiked with C. lytica) was added in triplicate to a 96-well plate. Additionally, 0.2 mL of Guillard's F/2 medium without any C. lytica or Formulation and 0.2 mL of Guillard's F/2 medium with C. lytica, but no Formulations, served as negative and positive growth controls, respectively. The 96-well plates were placed in an illuminated growth cabinet with a 16:8 light:dark cycle (photon flux density 33 μmol m⁻² s⁻¹) for 48 hrs at 18° C. and measured for chlorophyll fluorescence using a multi-well plate spectrophotometer (excitation: 360 nm; emission: 670 nm). The efficacy of each Formulation was measured by determining the percent reduction in diatom growth as a function of Formulation concentration. The results are shown in Table 5.

TABLE 5
Percent reduction in growth of Cellulophaga lytica
from serial dilutions of Formulations 1-9.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 0.00% | 0.00% | 0.00% | 10.06% | 19.53% | 26.33% | 27.51% | 36.98% |
| PEI600 | 0.00% | 0.00% | 0.00% | 0.00% | 6.80% | 15.00% | 20.51% | 31.43% |
| PEI10,000 | 0.00% | 0.00% | 0.00% | 0.00% | 9.90% | 16.99% | 27.45% | 52.67% |
| ETCS | 0.00% | 5.92% | 10.06% | 11.24% | 33.73% | 57.40% | 69.23% | 14.20% |
| TCS | 12.29% | 32.40% | 59.78% | 86.87% | 86.87% | 87.15% | 87.15% | 86.59% |
| Formulation 1 | 0.00% | 0.00% | 0.00% | 0.00% | 0.00% | 27.19% | 76.61% | 82.16% |
| Formulation 2 | 0.00% | 0.00% | 4.09% | 1.17% | 20.18% | 51.75% | 77.49% | 70.47% |
| Formulation 3 | 0.00% | 0.00% | 3.22% | 2.05% | 30.12% | 80.12% | 76.02% | 66.08% |
| Formulation 4 | 0.00% | 0.00% | 0.00% | 0.00% | 0.00% | 3.43% | 7.58% | 9.69% |
| Formulation 5 | 0.00% | 0.00% | 0.00% | 0.00% | 0.00% | 0.00% | 0.00% | 79.11% |
| Formulation 6 | 0.00% | 0.00% | 7.76% | 4.31% | 13.79% | 71.84% | 47.41% | 23.85% |
| Formulation 7 | 0.00% | 0.00% | 0.00% | 0.00% | 0.00% | 0.00% | 0.00% | 0.00% |
| Formulation 8 | 0.00% | 0.00% | 0.00% | 0.00% | 0.00% | 8.56% | 12.09% | 18.08% |
| Formulation 9 | 0.00% | 4.44% | 6.21% | 4.14% | 2.07% | 0.00% | 0.00% | 0.00% |

The results displayed in Table 5 show that many of the Formulations were biocidal toward C. lytica. Biocidel activity was dependent on polymer composition.

### Example 7

### Biocidal Activity of Formulations 1-9 Against Halomonas pacifica

In accordance with the MIC test, working solutions for each Formulation were prepared by dissolving 100 mg of each Formulation in 10 mL of methanol (Sigma-Aldrich) to generate a 10 mg/mL solution. Next, 10 ml of Guillard's F/2 medium was spiked with 200 μL of the 10 mg/mL Formulation solution to achieve a final concentration of 0.2 mg/mL.

A series of dilutions of H. pacifica were prepared by diluting a 0.03 OD₆₀₀ H. pacifica culture in Guillard's F/2 medium to generate concentrations of 100 μg/mL, 50 μg/mL, 25 μg/mL, 12.5 μg/mL 6.25 μg/mL, 3.13 μg/mL, 1.56 μg/mL and 0.78 μg/mL. 0.2 mL of each H. pacifica concentration was added in triplicate to a 96-well plate. Additionally, 0.2 mL of Guillard's F/2 medium without any H. pacifica or Formulation and 0.2 mL of Guillard's F/2 medium with H. pacifica, but no Formulations, served as negative and positive growth controls, respectively. The 96-well plates were placed in an illuminated growth cabinet with a 16:8 light:dark cycle (photon flux density 33 μmol m⁻² s⁻¹) for 48 hrs at 18° C. and measured for chlorophyll fluorescence using a multi-well plate spectrophotometer (excitation: 360 nm; emission: 670 nm). The efficacy of each Formulation was measured by determining the percent reduction in diatom growth as a function of Formulation concentration. The results are shown in Table 6.

TABLE 6
Percent reduction in growth of Halomonas pacifica
from serial dilutions of Formulations 1-9.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 7.01% | 11.31% | 16.08% | 18.95% | 21.18% | 22.61% | 18.95% | 5.25% |
| PEI600 | 0.00% | 0.00% | 0.00% | 0.00% | 0.00% | 0.00% | 0.00% | 0.00% |
| PEI10,000 | 0.00% | 0.00% | 0.00% | 0.00% | 0.00% | 0.00% | 3.11% | 13.02% |
| ETCS | 0.00% | 11.15% | 11.78% | 12.90% | 16.72% | 16.08% | 7.01% | 0.00% |
| TCS | 0.00% | 0.00% | 0.00% | 9.94% | 17.32% | 37.65% | 93.07% | 92.77% |
| Formulation 1 | 0.00% | 2.17% | 9.00% | 12.83% | 14.33% | 15.83% | 17.33% | 33.17% |
| Formulation 2 | 0.00% | 4.50% | 6.83% | 14.50% | 17.33% | 21.67% | 29.83% | 75.50% |
| Formulation 3 | 0.00% | 0.00% | 0.67% | 3.67% | 14.83% | 20.33% | 21.17% | 49.17% |
| Formulation 4 | 0.00% | 0.00% | 0.00% | 0.00% | 22.20% | 93.17% | 94.16% | 94.15% |
| Formulation 5 | 0.00% | 0.00% | 0.00% | 0.00% | 14.66% | 69.86% | 94.45% | 92.85% |
| Formulation 6 | 7.08% | 8.96% | 9.75% | 13.68% | 21.07% | 27.04% | 69.97% | 56.76% |
| Formulation 7 | 0.00% | 0.00% | 0.00% | 0.00% | 18.03% | 94.08% | 94.07% | 92.41% |
| Formulation 8 | 1.20% | 0.00% | 0.00% | 1.49% | 36.43% | 94.28% | 90.60% | 87.38% |
| Formulation 9 | 0.00% | 6.05% | 7.32% | 9.55% | 10.35% | 13.69% | 7.64% | 0.00% |

The results displayed in Table 6 show that most of the Formulations were biocidal toward H. pacifica. Biocidal activity was dependent on polymer composition.

### Example 8

### Biocidal Activity of Formulations 10-12 Against Pseudoalteromonas atlantica, Cellulophaga lytica, Cobetia marina, and Halomonas pacifica

The antimicrobial activity of Formulations 10-12, also derived from ETCS and PEI, was determined toward a suite of marine microorganisms, namely, Pseudoalteromonas atlantica, Cellulophaga lytica, Cobetia marina, and Halomonas pacifica. The antimicrobial activity of Formulations 10-12 was determined toward these marine microorganisms according to the methods of EXAMPLES 5-7. The MIC for each Formulation against each strain is compared to that of the PEI, ETCS, and TCS (alone) in Table 7.

TABLE 7
MIC results for Formulations 10-12 against Pseudoalteromonas atlantica,
Cellulophaga lytica, Cobetia marina, and Halomonas pacifica.

| | Microorganism | | | |
|---|---|---|---|---|
| | P. atlantica | C. lytica | C. marina | H. pacifica |
| Compound | MIC (μg/mL) | MIC (μg/mL) | MIC (μg/mL) | MIC (μg/mL) |
| PEI423 | >100 | 100 | >100 | >100 |
| ETCS | >100 | >100 | >100 | >100 |
| TCS | 50.0 | 6.25-12.5 | >100 | 50-100 |
| Formulation 10 | 100 | 50 | 50 | >100 |
| Formulation 11 | 25 | 50 | 50 | >100 |
| Formulation 12 | 25 | 25 | 25 | >100 |

“>100 μg/mL” indicates that no inhibition of growth was observed at a polymer concentration of 100 μg/mL. Thus, the MIC for these samples was greater than 100 μg/mL.

The results displayed in Table 7 show that Formulations 10-12 were biocidal towards P. atlantica, C. lytica, and C. marina. Surprisingly, Formulations 10-12 were active toward C. marina while TCS was not.

### Example 9

### Epoxy Trichlorophenol Polyethylenimine Formulations

The Formulations described in EXAMPLE 2 were reproduced by creating an epoxy trichlorophenol (ETCP) as in EXAMPLE 1, and then reacting the ETCP with a variety of polyethylenimines, as described in EXAMPLE 2.

Synthesis of ETCP—80 g of isopropanol and 19.4 g (0.346 mol) potassium hydroxide were charged to a one liter, three-neck, round-bottom flask equipped with a magnetic stir bar, and the mixture stirred at room temperature until the potassium hydroxide dissolved. In an 800 mL glass beaker, 100 g (0.5 mol) of 2,4,6-trichlorophenol (TCP) (Sigma-Aldrich) was dissolved at room temperature in 250 g of isopropanol using magnetic stirring. The TCP solution was subsequently added to the round-bottom flask containing the isopropanol/potassium hydroxide solution. The flask was then placed in a temperature-controlled silicone oil bath and equipped with a condenser and a 250 mL addition funnel. A thermocouple was placed into the reaction flask and the temperature controller set at 60° C.

Once the temperature had equilibrated, 140 g (1.5 mol) of epichlorohydrin was added dropwise to the solution over the course of 5 minutes using the addition funnel. During the course of the reaction, a precipitate (potassium chloride) was formed. The reaction was allowed to run for 16 hours. Upon completion of the reaction, the reaction mixture was transferred to a one-liter, single-neck, round-bottom flask and then placed in a rotary evaporator at reduced pressure for two hours to remove unreacted epichlorohydrin. Further purification was done using solvent extraction with water and a 1:1 mixture of hexanes and toluene. The organic phase was washed four times with water and dried over magnesium sulfate. Remaining solvent was removed at reduced pressure on a rotary evaporator and the clear viscous liquid product was collected (yield: 88%). The purified ETCP was characterized using proton nuclear magnetic resonance spectroscopy CH NMR) and high performance liquid chromatography (HPLC).

Synthesis of ETCP/polyethylenimine Formulations—10.9 grams of an oligomer mixture of polyethylenimine having an average Mₙ≈423 (PEI423) and 4.1 g of ETCP (prepared above) were dissolved at room temperature in 80 g of chloroform using a 250 mL single-neck, round-bottom flask designed for use with a Radley Six-Place Carousel Reactor System. The flask was placed in the Radley Six-Place Carousel Reactor System under a nitrogen blanket and condenser with temperature controller set at 50° C. The reaction was allowed to run for 64 hours and reaction progress was monitored using ¹H NMR. Upon completion of the reaction, solvent was then removed at reduced pressure using a rotary evaporator. The PEI423/ETCP polymer, a yellow viscous liquid, was collected and characterized using ¹H NMR and differential scanning calorimetry (DSC).

Formulations 69-77 were synthesized using the same synthetic procedure as above, with the exception that PEI composition (i.e. molecular weight) and ETCP concentration were varied. The ratios are shown below in TABLE 8.

TABLE 8
Compositions of Formulations 69-77.

| | PEI Used | Wt. PEI (g) | Wt. ETCP (g) |
|---|---|---|---|
| Formulation 69 | PEI423 | 10.9 | 4.1 |
| Formulation 70 | PEI600 | 10.9 | 4.1 |
| Formulation 71 | PEI10000 | 10.9 | 4.1 |
| Formulation 72 | PEI423 | 8.6 | 6.4 |
| Formulation 73 | PEI600 | 8.6 | 6.4 |
| Formulation 74 | PEI10000 | 8.6 | 6.4 |
| Formulation 75 | PEI423 | 6 | 9 |
| Formulation 76 | PEI600 | 6 | 9 |
| Formulation 77 | PEI10000 | 6 | 7.1 |

### Example 10

### Comparison of TCP Polyethylenimine Polymeric Biocides to Benzylpiperidino Biocides

For comparison purposes a benzylpiperidino biocide (“Reference 1”) was prepared using ETCS and synthetic methods similar to those described in U.S. Pat. No. 5,036,077, issued Jul. 30, 1991, and incorporated by reference in its entirety. To make Reference 1, 3.9 g of ETCS and 2 g of 4-benzylpiperidine were reacted in 30 mL of methanol. The mixture was heated at 65° C. for 48 hours. Reaction completion was confirmed by ¹H NMR, ¹³C NMR, HMQC (2D) NMR and elemental analysis. The antimicrobial activity of Formulations 69-77 and Reference 1 toward S. epidermidis was measured by determining the percent reduction in bacterial growth as a function of concentration, as was done above in EXAMPLE 3. The results obtained are shown in TABLE 9.

TABLE 9
Percent reduction in growth of Staphylococcus epidermidis
from Formulations 69-77 and Reference 1.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 0.00% | 12.50% | 14.55% | 24.32% | 24.20% | 18.52% | 24.77% | 8.98% |
| PEI600 | 19.65% | 23.52% | 25.23% | 26.44% | 28.25% | 26.63% | 20.33% | 53.42% |
| PEI10,000 | 13.11% | 19.65% | 23.44% | 19.07% | 20.92% | 17.99% | 16.02% | 63.11% |
| EDCP | 7.58% | 6.34% | 5.04% | 8.44% | 6.20% | 8.90% | 5.00% | 0.00% |
| Formulation 69 | 1.04% | 5.87% | 6.52% | 4.83% | 1.92% | 4.79% | 99.54% | 99.25% |
| Formulation 70 | 0.00% | 2.86% | 2.95% | 1.38% | 2.79% | 99.04% | 99.68% | 99.13% |
| Formulation 71 | 1.10% | 4.97% | 4.25% | 3.18% | 4.47% | 14.91% | 93.90% | 97.04% |
| Formulation 72 | 5.80% | 11.28% | 11.74% | 10.27% | 62.21% | 99.74% | 99.74% | 99.25% |
| Formulation 73 | 2.02% | 4.17% | 4.43% | 6.13% | 99.29% | 99.13% | 99.04% | 96.50% |
| Formulation 74 | 2.33% | 6.48% | 4.37% | 4.65% | 4.18% | 93.43% | 88.08% | 90.41% |
| Formulation 75 | 3.75% | 9.62% | 31.86% | 76.04% | 100.00% | 100.00% | 99.84% | 97.59% |
| Formulation 76 | 3.75% | 4.84% | 7.35% | 44.91% | 99.62% | 98.62% | 95.00% | 88.35% |
| Formulation 77 | 3.96% | 5.25% | 6.67% | 7.04% | 5.44% | 34.65% | 87.74% | 79.69% |
| Reference 1 | 3.12% | 8.01% | 6.34% | 14.09% | 21.78% | 30.44% | 33.98% | 31.92% |

The results shown in TABLE 9 show that the polymers derived from ETCP and PEI are antimicrobial toward S. epidermidis and the antimicrobial activity is a function of polymer composition. Formulation 75 displayed the highest antimicrobial activity. All of the Formulations showed higher antimicrobial activity compared to Reference 1.

Next, the antimicrobial activity of Formulations 69-77 and Reference 1 toward E. coli was measured by determining the percent reduction in bacterial growth as a function of concentration. The results for the examples were compared to various PEIs used to produce the examples. The results obtained are shown in TABLE 10.

TABLE 10
Percent reduction in growth of Escherichia coli
from Formulations 69-77 and Reference 1.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 0.00% | 0.00% | 0.00% | 0.00% | 0.00% | 2.85% | 6.49% | 0.00% |
| PEI600 | 0.00% | 4.57% | 0.00% | 2.47% | 6.27% | 11.12% | 20.23% | 22.12% |
| PEI10,000 | 0.00% | 7.38% | 7.25% | 6.90% | 26.70% | 29.81% | 57.07% | 59.61% |
| EDCP | 14.85% | 1.60% | 0.00% | 55.43% | 52.95% | 61.26% | 1.38% | 91.65% |
| Formulation 69 | 4.87% | 2.35% | 0.97% | 0.00% | 3.42% | 2.95% | 25.50% | 42.58% |
| Formulation 70 | 0.00% | 0.00% | 0.00% | 0.00% | 0.00% | 14.73% | 31.23% | 57.01% |
| Formulation 71 | 0.00% | 0.00% | 0.00% | 0.00% | 0.00% | 0.00% | 37.63% | 34.70% |
| Formulation 72 | 0.00% | 0.00% | 2.42% | 0.00% | 0.00% | 0.00% | 46.01% | 98.79% |
| Formulation 73 | 0.00% | 0.00% | 0.00% | 0.00% | 0.00% | 10.03% | 39.08% | 78.47% |
| Formulation 74 | 0.00% | 0.00% | 0.00% | 0.00% | 0.00% | 0.00% | 0.11% | 8.57% |
| Formulation 75 | 0.00% | 0.74% | 1.81% | 4.19% | 3.49% | 99.66% | 99.09% | 96.74% |
| Formulation 76 | 0.00% | 0.00% | 0.00% | 0.00% | 0.00% | 0.00% | 59.56% | 82.60% |
| Formulation 77 | 0.00% | 2.22% | 15.34% | 0.00% | 0.00% | 0.00% | 0.00% | 0.00% |
| Reference 1 | 0.00% | 0.00% | 0.00% | 0.00% | 0.00% | 0.00% | 0.00% | 0.00% |

The results shown in TABLE 10 show that many of the polymers derived from ETCP and PEI are antimicrobial toward E. coli and antimicrobial activity is a function of polymer composition. Formulation 75 displayed the highest antimicrobial activity. Reference 1 showed no antimicrobial activity toward E. coli.

Additionally, the antifungal activity of Formulations 69-77 and Reference 1 toward Candida albicans was measured by determining the percent reduction in fungal growth as a function of concentration. The results obtained are shown in TABLE 11.

TABLE 11
Percent reduction in growth of Candida albicans
from Formulations 69-77 and Reference 1.

| | Formulation Concentration | | | | | | | |
|---|---|---|---|---|---|---|---|---|
| | 0.78125 | 1.5625 | 3.125 | 6.25 | 12.5 | 25 | 50 | 100 |
| Compound | (μg/mL) | (μg/mL) | (μg/mL) | (μg/mL) | (μg/mL) | (μg/mL) | (μg/mL) | (μg/mL) |
| PEI423 | 9.95% | 3.06% | 3.44% | 0.00% | 3.07% | 9.26% | 14.28% | 78.42% |
| PEI600 | 4.90% | 14.51% | 9.10% | 7.06% | 21.14% | 73.48% | 96.22% | 98.35% |
| PEI10,000 | 3.05% | 9.10% | 9.98% | 11.45% | 7.93% | 22.08% | 55.97% | 81.86% |
| EDCP | 11.66% | 15.25% | 0.00% | 1.45% | 0.00% | 0.00% | 21.55% | 25.83% |
| Formulation 69 | 2.12% | 1.63% | 2.19% | 4.72% | 6.17% | 12.22% | 11.32% | 32.59% |
| Formulation 70 | 1.19% | 1.65% | 3.73% | 8.45% | 38.42% | 96.30% | 98.71% | 99.29% |
| Formulation 71 | 1.96% | 3.21% | 3.67% | 8.67% | 2.93% | 13.72% | 49.06% | 90.92% |
| Formulation 72 | 2.22% | 3.77% | 3.70% | 3.85% | 38.71% | 10.48% | 17.90% | 98.65% |
| Formulation 73 | 2.49% | 5.33% | 4.92% | 9.16% | 12.38% | 51.56% | 99.47% | 98.63% |
| Formulation 74 | 2.47% | 5.18% | 3.09% | 3.37% | 4.59% | 13.26% | 24.58% | 97.14% |
| Formulation 75 | 3.67% | 3.85% | 6.81% | 11.76% | 3.42% | 85.28% | 99.01% | 96.94% |
| Formulation76 | 2.89% | 4.57% | 3.98% | 11.56% | 3.60% | 99.47% | 98.86% | 97.79% |
| Formulation 77 | 5.12% | 4.21% | 3.62% | 5.25% | 2.70% | 6.09% | 16.78% | 14.94% |
| Reference 1 | 2.55% | 2.11% | 0.00% | 0.21% | 18.24% | 5.18% | 88.68% | 99.06% |

The results shown in TABLE 11 show that some of the polymers derived from ETCP and PEI possess good antimicrobial activity toward C. albicans. Formulations 70, 75, and 76 displayed the highest antimicrobial activity.

### Example 11

### Biocidal Resins

In addition to PEI-based biocidal polymers, ETCS can be reacted with a variety of amines to form compounds with antimicrobial activity. Several Formulations are described below. The efficacy of the resins against various microorganisms is shown in TABLE 12.

Formulation 13: 5.0 g of bis(2-methoxyethyl)amine (Sigma-Aldrich) and 12.9 g of ETCS were dissolved at room temperature in 80 g of chloroform using a 250 mL single-neck, round-bottom flask designed for use with a Radley Six-Place Carousel Reactor System. The flask was placed in the Radley Six-Place Carousel Reactor System under a nitrogen blanket and condenser with temperature controller set at 50° C. The reaction was allowed to run for 64 hours and reaction progress was monitored using ¹H NMR. Upon completion of the reaction, solvent was then removed at reduced pressure using a rotary evaporator. The product, a yellow viscous liquid, was collected and characterized using ¹H NMR.

Formulations 14-20 were synthesized using the same synthetic procedure as was done for Formulation 13 with the exception that the composition of the amine and the concentration of ETCS was varied.

Formulation 14: 5.0 g of diethylamine (Sigma-Aldrich) and 11.8 g of ETCS were dissolved at room temperature in 80 g of chloroform using a 250 mL single-neck, round-bottom flask designed for use with a Radley Six-Place Carousel Reactor System.

Formulation 15: 10.0 g of N-ethyl-2-methylallyl amine (Sigma-Aldrich) and 17.4 g of ETCS were dissolved at room temperature in 80 g of chloroform using a 250 mL single-neck, round-bottom flask designed for use with a Radley Six-Place Carousel Reactor System.

Formulation 16: 5.0 g of diisopropanolamine (Sigma-Aldrich) and 13.0 g of ETCS were dissolved at room temperature in 80 g of chloroform using a 250 mL single-neck, round-bottom flask designed for use with a Radley Six-Place Carousel Reactor System.

Formulation 17: 3.0 g of diethanolamine (Sigma-Aldrich) and 9.9 g of ETCS were dissolved at room temperature in 80 g of chloroform using a 250 mL single-neck, round-bottom flask designed for use with a Radley Six-Place Carousel Reactor System.

Formulation 18: 3.0 g of N-methylaniline (Sigma-Aldrich) and 9.8 g of ETCS were dissolved at room temperature in 80 g of chloroform using a 250 mL single-neck, round-bottom flask designed for use with a Radley Six-Place Carousel Reactor System.

Formulation 19: 1.1 g of ethylenediamine (Sigma-Aldrich) and 11.5 g of ETCS were dissolved at room temperature in 80 g of chloroform using a 250 mL single-neck, round-bottom flask designed for use with a Radley Six-Place Carousel Reactor System.

Formulation 20:1.0 g of allyl amine (Sigma-Aldrich) and 6.2 g of ETCS were dissolved at room temperature in 80 g of chloroform using a 250 mL single-neck, round-bottom flask designed for use with a Radleys Six-Place Carousel Reactor System.

TABLE 12
MIC results for Formulations 13-20 and precursors used to make them.

| | Microorganism | | | | |
|---|---|---|---|---|---|
| | C. lytica | H. pacifica | N. incerta | E. Coli | S. epidermidis |
| Compound | MIC (μg/mL) | MIC (μg/mL) | MIC (μg/mL) | MIC (μg/mL) | MIC (μg/mL) |
| ETCS | >100 | >100 | >100 | >100 | >100 |
| TCS | 12.5 | 100 | 3.125 | 0.781 | 0.781 |
| allyl amine | >100 | >100 | >100 | >100 | >100 |
| diisopropanolamine | >100 | >100 | >100 | >100 | >100 |
| diethanolamine | >100 | >100 | >100 | >100 | >100 |
| N-methyl aniline | >100 | >100 | >100 | >100 | >100 |
| ethylenediamine | >100 | >100 | >100 | >100 | >100 |
| bis(methoxyethyl) | >100 | >100 | >100 | >100 | >100 |
| Formulation 13 | >100 | >100 | >100 | >100 | 12.5 |
| Formulation 16 | 50 | >100 | 50 | >100 | 12.5 |
| Formulation 17 | 50 | >100 | 25 | >100 | 50 |
| Formulation 18 | >100 | >100 | 100 | >100 | 100 |
| Formulation 19 | >100 | >100 | 12.5 | 12.5 | 50 |
| Formulation 20 | 50 | >100 | 25 | >100 | 100 |

“>100 μg/mL” indicates that no inhibition of growth was observed at a biocide concentration of 100 μg/mL. Thus, the MIC for these samples was greater than 100 μg/mL.

The results displayed in TABLE 12 show that some of the Formulations exhibit biocidel activity toward several of the microorganisms.

### Example 12

### Antimicrobial Coatings Comprising ETCS

Polymeric coatings may be derived from various PEI-based polymers produced in the preceding EXAMPLES by creating crosslinked networks that incorporate multifunctional epoxides. The coatings can be deposited in 24-well polystyrene plates modified with aluminum discs in the bottom of each well, and the antimicrobial properties of the coatings determined using an algal biofilm growth assay described below.

An array of PEI-based polymers derived from PEI423, ETCS, iodooctane (Sigma-Aldrich), and a monofunctional epoxy siloxane (MW=1000) (Dow Corning, Midland, Mich.) was synthesized in a SYMYX BATCH REACTOR SYSTEM™ (Symyx Technologies, Inc., Sunnyvale, Calif.). Automated dispensing of varying amounts of PEI, ETCS, iodooctane, and epoxy siloxane was done into a 2×6 array of 8 mL glass vials containing magnetic stir bars using the SYMYX BATCH REACTOR SYSTEM™. Reagent addition was followed by the addition of chloroform to create 30% by weight solutions. TABLE 13 provides the composition of each reaction mixture generated. After the addition of the reagents, the vials were sealed, stirring was initiated, and the reaction mixtures heated at 50° C. for 40 hours. The resulting polymer array was characterized using nuclear magnetic spectroscopy (NMR) and differential scanning calorimetry (DSC).

TABLE 13
Composition of reaction mixtures used
to prepare Formulations 21-32.

| | PEI423 | ETCS | Epoxy | Iodooc- | Chloro- |
|---|---|---|---|---|---|
| Formulation | (mg) | (mg) | PDMS (mg) | tane (mg) | form (mg) |
| Formulation 21 | 959 | 1072 | 64 | 38 | 6397 |
| Formulation 22 | 959 | 1072 | 64 | 38 | 6397 |
| Formulation 23 | 637 | 1294 | 129 | 93 | 6459 |
| Formulation 24 | 959 | 1072 | 64 | 38 | 6397 |
| Formulation 25 | 959 | 1072 | 64 | 38 | 6397 |
| Formulation 26 | 1864 | 189 | 0 | 0 | 6162 |
| Formulation 27 | 1735 | 176 | 123 | 15 | 6148 |
| Formulation 28 | 714 | 1452 | 0 | 0 | 6498 |
| Formulation 29 | 1853 | 188 | 0 | 13 | 6164 |
| Formulation 30 | 683 | 1387 | 0 | 96 | 6499 |
| Formulation 31 | 1748 | 178 | 123 | 0 | 6145 |
| Formulation 32 | 667 | 1356 | 129 | 0 | 6458 |

Coatings were produced from Formulations 21-32 using a SYMYX™ coating formulation system by solution blending the mixtures with neopentyl glycol diglycidyl ether (Sigma-Aldrich). TABLE 14 lists the composition of each coating solution prepared. After allowing the coating solutions to stir briefly to ensure homogeneity, coatings were deposited into 24-well polystyrene plates modified with aluminum discs in the bottom of each well. The aluminum discs were primed with Intergard 264 (International Paint, Houston, Tex.) to ensure good adhesion of the coatings to the discs. The antimicrobial properties of the coatings were determined using the marine microorganism, Navicula incerta (diatom algae), and two biological assays, namely, a leachate toxicity assay and a biofilm growth assay.

TABLE 14
Compositions of coating solutions for Formulations 33-68.

| | | Weight of PEI- | Neopentyl glycol |
|---|---|---|---|
| | PEI-based | based polymer | diglycidyl |
| Formulation | polymer | solution (mg) | ether (mg) |
| Formulation 33 | Formulation 21 | 3384 | 644 |
| Formulation 34 | Formulation 21 | 3166 | 804 |
| Formulation 35 | Formulation 21 | 2974 | 944 |
| Formulation 36 | Formulation 22 | 3384 | 644 |
| Formulation 37 | Formulation 22 | 3166 | 804 |
| Formulation 38 | Formulation 22 | 2974 | 944 |
| Formulation 39 | Formulation 23 | 3730 | 417 |
| Formulation 40 | Formulation 23 | 3571 | 533 |
| Formulation 41 | Formulation 23 | 3425 | 639 |
| Formulation 42 | Formulation 24 | 3384 | 644 |
| Formulation 43 | Formulation 24 | 3166 | 804 |
| Formulation 44 | Formulation 24 | 2974 | 944 |
| Formulation 45 | Formulation 25 | 3384 | 644 |
| Formulation 46 | Formulation 25 | 3166 | 804 |
| Formulation 47 | Formulation 25 | 2974 | 944 |
| Formulation 48 | Formulation 26 | 2626 | 1126 |
| Formulation 49 | Formulation 26 | 2344 | 1340 |
| Formulation 50 | Formulation 26 | 2117 | 1512 |
| Formulation 51 | Formulation 27 | 2689 | 1073 |
| Formulation 52 | Formulation 27 | 2413 | 1283 |
| Formulation 53 | Formulation 27 | 2188 | 1454 |
| Formulation 54 | Formulation 28 | 3687 | 464 |
| Formulation 55 | Formulation 28 | 3513 | 590 |
| Formulation 56 | Formulation 28 | 3355 | 704 |
| Formulation 57 | Formulation 29 | 2632 | 1121 |
| Formulation 58 | Formulation 29 | 2351 | 1335 |
| Formulation 59 | Formulation 29 | 2124 | 1508 |
| Formulation 60 | Formulation 30 | 3712 | 446 |
| Formulation 61 | Formulation 30 | 3543 | 568 |
| Formulation 62 | Formulation 30 | 3389 | 679 |
| Formulation 63 | Formulation 31 | 2681 | 1078 |
| Formulation 64 | Formulation 31 | 2404 | 1289 |
| Formulation 65 | Formulation 31 | 2179 | 1460 |
| Formulation 66 | Formulation 32 | 3706 | 435 |
| Formulation 67 | Formulation 32 | 3541 | 554 |
| Formulation 68 | Formulation 32 | 3391 | 663 |

For the leachate toxicity assay, coating arrays were immersed in a recirculating water bath for 2 weeks to remove leachable residues from the coatings. The preconditioned coatings were then incubated in 1 ml of growth medium for 24 hrs and the resultant coating leachates collected. Then, 0.05 ml of a N. incerta suspension in Guillard's F/2 medium (˜10⁵ cells ml⁻¹) was added to 1 ml of coating leachate and 0.2 ml of the coating leachate with the added microorganism was transferred in triplicate to a 96-well array plate. The coating array plates were incubated for 48 hrs at 18° C. in an illuminated growth cabinet with a 16:8 light:dark cycle (photon flux density 33 μmol m⁻² s⁻¹) for N. incerta. N. incerta-containing array plates were characterized by extracting biofilms with dimethyl sulfoxide and quantifying chlorophyll concentration using fluorescence spectroscopy (excitation: 360 nm; emission: 670 nm). A reduction in the amount of algal growth compared with a positive growth control (i.e., organism in fresh growth media) was considered to be a consequence of toxic components being leached from the coating into the overlying medium. The biofilm growth assay was completed as described in EXAMPLE 5.

None of the coating leachates showed any toxicity indicating that they were not leaching toxic components. The results of biofilm growth assay are shown in TABLE 15. The results shown in TABLE 15 indicate that many of the coatings inhibit biofilm growth of N. incerta.

TABLE 15
Antimicrobial properties of Formulations 33-68.

| | Reduction in Biofilm |
|---|---|
| Formulation | Growth for N. incerta |
| Formulation 33 | 92% |
| Formulation 34 | 78% |
| Formulation 35 | 62% |
| Formulation 36 | 93% |
| Formulation 37 | 57% |
| Formulation 38 | 66% |
| Formulation 39 | 82% |
| Formulation 40 | 0% |
| Formulation 41 | 0% |
| Formulation 42 | 85% |
| Formulation 43 | 0% |
| Formulation 44 | 0% |
| Formulation 45 | 80% |
| Formulation 46 | 0% |
| Formulation 47 | 0% |
| Formulation 48 | 79% |
| Formulation 49 | 0% |
| Formulation 50 | 0% |
| Formulation 51 | 76% |
| Formulation 52 | 0% |
| Formulation 53 | 0% |
| Formulation 54 | 29% |
| Formulation 55 | 0% |
| Formulation 56 | 0% |
| Formulation 57 | 86% |
| Formulation 58 | 0% |
| Formulation 59 | 0% |
| Formulation 60 | 14% |
| Formulation 61 | 0% |
| Formulation 62 | 0% |
| Formulation 63 | 91% |
| Formulation 64 | 0% |
| Formulation 65 | 0% |
| Formulation 66 | 78% |
| Formulation 67 | 51% |
| Formulation 68 | 0% |

### Prophetic Examples

### Example 13

### Cosmetic Lotion Comprising ETCS/PEI Formulation 5

The following ingredients will be mixed to form a lotion comprising ETCS/PEI Formulation 5 as described in EXAMPLE 2.

| Ingredient | w/w percent |
|---|---|
| cetyl alcohol | 0.75 |
| C12-15 alcohols benzoate | 5 |
| butylated hydroxyanisole | 0.1 |
| PEG-100 stearate | 0.25 |
| water, deionized or distilled | 79.4 |
| propylene glycol | 3.0 |
| Formulation 5 (Ex. 2) | 1.0 |
| acetone | 10.0 |

### Example 14

### Cosmetic Cream Comprising ETCS/PEI Formulation 5

The following ingredients will be mixed to form a cream comprising an ETCS/PEI Formulation 5 as described in EXAMPLE 2.

| Ingredients | w/w percent |
|---|---|
| Cetyl-stearyl alcohol | 2.25 |
| C12-15 alcohol benzoate | 5 |
| Butylated hydroxyanisole | 0.01 |
| PEG-100 stearate | 0.85 |
| Water, deionized or distilled | 78 |
| Propylene glycol | 3 |
| Formulation 5 (Ex. 2) | 1.0 |
| Acetone | 10 |

### Example 15

### Mascara Comprising ETCS/PEI Formulation 1

The following ingredients will be mixed to form black, brown, blue, and green mascaras comprising an ETCS/PEI Formulation 1 as described in EXAMPLE 2.

| | Black | Brown | Blue | Green |
|---|---|---|---|---|
| Component | (wt %) | (wt %) | (wt %) | (wt %) |
| Water | 86.87 | 87.34 | 86.34 | 85.70 |
| SD Alcohol 40-B | 4.50 | 4.50 | 4.50 | 4.50 |
| TEA-Carbomer 940 | 3.28 | 3.30 | 3.30 | 2.94 |
| Polyvinyl Alcohol | 1.00 | 1.00 | 1.00 | 1.00 |
| Glycerin | 0.50 | 0.50 | 0.50 | 0.50 |
| Methylparaben | 0.25 | 0.25 | 0.25 | 0.25 |
| Imidazolidinyl Urea | 0.01 | 0.01 | 0.01 | 0.01 |
| Trisodium EDTA | 0.05 | 0.05 | 0.05 | 0.05 |
| Hydrolyzed Animal Protein | 0.05 | 0.05 | 0.05 | 0.05 |
| Lecithin-treated Pigments | | | | |
| Chromiun Hydroxide Green | | | | 4.00 |
| Iron Oxide Black | 2.50 | 0.60 | | |
| Iron Oxide Yellow | | 0.70 | | |
| Iron Oxide Red | | 0.70 | | |
| Ultramarine Blue | | | | 3.00 |
| Formulation 1 | 1.00 | 1.00 | 1.00 | 1.00 |

### Example 16

### Toothpaste Comprising ETCS/PEI Formulation 5

The following ingredients will be mixed to form a toothpaste comprising an ETCS/PEI Formulation 5 as described in EXAMPLE 2.

| | Ingredients | % w/w |
|---|---|---|
| A | Sorbitol USP | 17.0 |
| | Formulation 5 (Ex. 2) | 2.5 |
| B | Glycerin USP 96% | 11.0 |
| | Na-Saccharin USP 40/60 Mesh | 0.2 |
| | Veegum D-Granular | 2.0 |
| | Peppermint Oil | 1.1 |
| | Stepanol WA/100 (Na-Lauryl Sulfate) | 2.2 |
| C | Veegum HF-6% (Ag/Al Silicate) | 16.64 |
| | Blue #1 FD + C (0.6%) | 0.06 |
| D | Na-CMC 7 H 5% | 47.3 |

### Example 17

### Survival Rates for Clostridium difficile on Formulation 63

A 10 mm×10 mm aluminum test plate will be covered with Formulation 63 (“sample”). The sample will be degreased and cleaned by vortexing the sample in ethanol. As a control, a 10 mm×10 mm piece of 3 mm thick stainless steel (“control”) will also be degreased and immersed in ethanol, and the excess ethanol burned off.

Clostridium difficile on glycerol protected beads (Fisher Scientific) will be incubated anaerobically with brain heart infusion broth (Oxoid) at 37° C. for 3-5 days to produce a culture of vegetative cells and spores for testing. Both the control and sample will have 20 μL of the Clostridium difficile culture pipetted onto their respective surfaces, and the control and sample will be incubated at room temperature for 2 hours. After two hours of incubation, 20 μL of a 5 mM solution of CTC (5-Cyano-2,3-ditolyl tetrazolium chloride; Sigma-Aldrich) will be deposited on the sample and the control, and the sample and control will be incubated in a dark, humid chamber for at 37° C. for 8 hours.

After rinsing the sample and control with sterile DI water to remove excess CTC stain, the sample and control will be imaged using epifluorescent microscopy, and a series of field views will be collected with a digital camera. A count of cells or spores in these field views will show that after two hours of incubation, the control sample had a great number of metabolically active cells or spore (e.g., CTC-stained) while the sample had less than 1% of the metabolically active cells or spores that were found on the control. The data will thus confirm that the surfaces of Formulation 63 inhibit the growth of Clostridium difficile.

### Example 18

### Survival Rates for Listeria monocytogenes on Formulation 63

As in EXAMPLE 17, a 10 mm×10 mm aluminum test plate will be covered with Formulation 63 (“sample”). The sample will be degreased and cleaned by vortexing the sample in ethanol. As a control, a 10 mm×10 mm piece of 3 mm thick stainless steel (“control”) will also be degreased and immersed in ethanol, and the excess ethanol burned off.

Listeria monocytogenes Scott A from previously frozen microbeads (Centre for Applied Microbiology Research, Porton Down, UK) will be incubated with brain heart infusion broth (Oxoid) at 37° C. for 15-20 hours to produce an active culture for testing. Both the control and sample will have 20 μL of the Listeria monocytogenes culture pipetted onto their respective surfaces, and the control and sample will be incubated at room temperature for 2 hours. After two hours of incubation, 20 μL of a 5 mM solution of CTC (5-Cyano-2,3-ditolyl tetrazolium chloride; Sigma-Aldrich) will be deposited on the sample and the control, and the sample and control will be incubated in a dark, humid chamber for at 37° C. for 2 hours.

After rinsing the sample and control with sterile DI water to remove excess CTC stain, the sample and control will be imaged using epifluorescence microscopy, and a series of field views will be collected with a digital camera. A count of cells or in these field views will show that after two hours of incubation, the control sample had a great number of metabolically active cells (e.g., CTC-stained) while the sample had less than 1% of the metabolically active cells that were found on the control. The data will thus confirm that FORMULATION 63 inhibits the growth of Listeria monocytogenes.

### Example 19

### Survival Rates for Bacteria on Bathroom Handrails

Two commercial ADA-compliant stainless steel handrails (“commercial handrail”) will be cleaned with acetone and ethanol. One handrail will be coated with Formulation 63 (“test handrail”). The test handrail will be installed in a stall of a men's bathroom at an international airport. An adjoining stall, having a commercial handrail will be selected as the control. At 5:00 AM, both the test and commercial handrails will be thoroughly disinfected with a bleach solution, and rinsed with clean water. At 10:00 PM, after a full day of use, both handrails will be carefully removed from the stalls and bagged to prevent additional contamination.

The handrails will be taken to a laboratory, where the handrails will be sprayed with a 5 mM solution of CTC (5-Cyano-2,3-ditolyl tetrazolium chloride; Sigma-Aldrich) under low-light conditions, and then allowed to incubate at 37° C. for 2 hours. After incubation, both handrails will be rinsed with sterile DI water. After air-drying, an ultraviolet lamp will be used to assess the fluorescence on both handrails, the fluorescence being indicative of the presence of active bacteria. The commercial handrail will show a substantially greater amount of fluorescence, indicating that after a full day of use, the test handrail had substantially fewer active bacteria on its surface.

Thus, the invention provides, among other things, a polymeric biocide and compositions and coatings comprising a polymeric biocide. Various features and advantages of the invention are set forth in the following claims.

1. A polymeric biocide of formula (I): 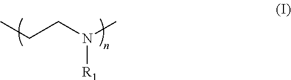
wherein each R₁ is independently H,
formula (II): 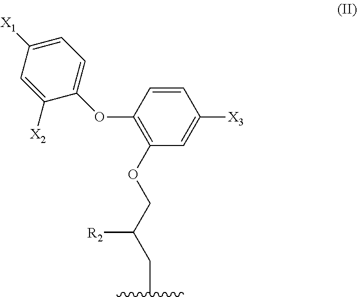
wherein X₁, X₂, X₃, are independently H or halogen and R₂ is OH, NHOH, NH₂, or C₁-C₄ alkyl alcohol,
formula (III): 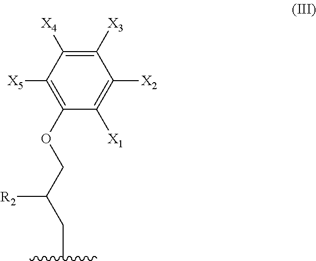
wherein X₁-X₅ are independently H or halogen and R₂ is OH, NHOH, NH₂, or C₁-C₄ alkyl alcohol,
or (CH₂)₂NR₃R₄ wherein R₃ and R₄ are independently H, (CH₂)₂NH₂, formula (II) or formula (III); and
n=5-50,000, wherein at least one R₁ or R₃ or R₄ is either formula (II) or formula (III).

2. The polymeric biocide of claim 1, wherein X₁, X₂, X₃, X₄, or X₅ is chlorine.

3. A lotion comprising the polymeric biocide of claim 1.

4. A cosmetic comprising the polymeric biocide of claim 1.

5. The cosmetic of claim 4, selected from the group consisting of mascara, foundation, blush, lipstick, and eye shadow, eyeliner, concealer, wrinkle cream, and moisturizer.

6. The polymeric biocide of claim 1, further comprising a cross-linker.

7. The polymeric biocide of claim 6, wherein the cross-linker is a hydrophilic compound with functional groups capable of reacting with amines.

8. The polymeric biocide of claim 7, wherein the functional groups are epoxides or isocyanates.

9. A hydrogel comprising the polymeric biocide of claim 7.

10. A coating comprising the polymeric biocide of claim 1.

11. The coating of claim 10, wherein the coating is a marine paint.

12. An article coated with coating of claim 10, wherein the article is selected from the group consisting of knobs, handles, rails, poles, countertops, sinks, and faucets.

13. A medical device coated with the polymeric biocide of claim 1.

14. An antimicrobial composition comprising the polymeric biocide of claim 1.

15. The antimicrobial composition of claim 14, further comprising an emulsifier, a stabilizer, a thickener, a humectant, a plasticizer, a fragrance, a pigment, or a dye.

16. The antimicrobial composition of claim 14, further comprising an additional polymer.

17. The antimicrobial composition of claim 14, further comprising a cross-linker.

18. A method of reducing the growth of a microorganism, comprising contacting the microorganism with a polymeric biocide of claim 1.

19. The method of claim 18, wherein the microorganism is selected from the group consisting of Staphylococcus epidermidis, Escherichia coli, Navicula incerta, Cellulophaga lytica, Halomonas pacifica, Pseudoalteromonas atlantica, Cobetia marina, Candida albicans, Clostridium difficile, Listeria monocytogenes.

20. A method of reducing the growth of a microorganism on a surface, comprising coating a surface to have reduced growth of a microorganism with a polymeric biocide of claim 1.

21. The method of claim 20, wherein the surface is selected from the group consisting of medical surfaces, marine surfaces, and household surfaces.

22. A method of reducing the formation of a biofilm, comprising coating a surface to be protected from the biofilm with a polymeric biocide of claim 1.
